(19) United States
(12) Patent Application Publication
(10) Pub. No.: US 2024/0225568 A9
(43) Pub. Date: Jul. 11, 2024

(54) ENHANCED INVASIVE IMAGING SYSTEMS AND METHODS

(71) Applicants: James Cassuto, Chatham, NJ (US); Mark Ferretti, Mountain Top, PA (US)

(72) Inventors: James Cassuto, Chatham, NJ (US); Mark Ferretti, Mountain Top, PA (US)

(21) Appl. No.: 17/973,468

(22) Filed: Oct. 25, 2022

(51) Int. Cl. A61B 6/03; G06T 7/00

(52) U.S. Cl. CPC A61B 6/037; G06T 7/0012; G06T 2207/10

(57) ABSTRACT

Computer-implemented enhanced invasive imaging methods, systems, and computer-readable media are described.

### FIELD

Some implementations generally relate to medical imaging systems, and, in particular, to enhanced invasive study imaging systems that combine non-invasive study data and invasive study data in real time to provide an enhanced display and automatically generated suggestions of potential areas of interest and/or elevated risk.

### BACKGROUND

Some conventional invasive study imaging systems may not provide enhanced invasive study images in real-time (or near real-time) that include imagery or symbology based on a fusion of non-invasive imaging study data and invasive imaging study data. Further some conventional system may not suggest regions of potential interest or regions of elevated risk based on non-invasive data and/or predict regions of potential interest or elevated risk based on non-invasive study data processed by a machine learning model to predict potential risk areas not associated only with hard plaques.

The background description provided herein is for the purpose of presenting generally the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

Some implementations can include a computer-implemented method. The method can include obtaining one or more first images generated by a first imaging modality and detecting one or more first regions of interest based on a first detection modality. The method can also include detecting one or more second regions of interest based on a second detection modality, and determining a location of imaging within a subject body in a second imaging modality

The method can further include generating second region of interest symbology indicating the one or more second regions of interest based on the location of imaging within the subject body in the second imaging modality. The method can also include combining the second region of interest symbology with one or more invasive study images generated by the second imaging modality to generate one or more enhanced invasive study images and causing the one or more enhanced invasive study images to be displayed during an invasive imaging study of the second imaging modality.

In some implementations, the first imaging (or detection) modality includes positron emission tomography (PET) or hybrid PET-CT (computed tomography), and wherein the one or more first images include positron emission tomography images. In some implementations, the one or more first regions of interest each include an area containing a suspected hard plaque. In some implementations, the one or more second regions of interest each include an area containing a suspected soft plaque. In some implementations, the one or more second regions of interest can each include an area containing a suspect plaque with associated functional PET data showing active inflammation, increased metabolism, instability, or increased risk of rupture. PET can often provide the most information about soft plaque risk of rupture compared to other imaging modalities. Other modalities that may be routinely used and could be used to gather a similar risk profile for a region of interest could be CT and MRI. PET includes functional information and hybrid PET-CT provides the functional information overlayed onto a CT, which helps with anatomic localization. In the early 1990s, isolated PET imaging was common. Currently, PET-CT is common, even though for the purposes of using PET we are interested in the PET data. CT can show microcalcifications in plaques which could mean increased risk of rupture. MRI can show high T1 signal which could mean intraplaque hemorrhage. In some implementations, a hybrid PET-MRI could also be used where regions of high inflammation from PET and high T1 signal from MRI could synergistically be more predictive of risk of rupture.

In some implementations, the second imaging (or detection) modality includes a machine learning model trained to predict regions within an artery containing a suspected soft plaque in particular a plaque with increased risk of rupture.

In some implementations, generating the second region of interest symbology indicating the one or more second regions of interest based on the location of imaging within the subject body in the second imaging modality includes aligning a location of the one or more second regions of interest with the location of imaging within the subject body, and associating each element of the second region of interest symbology with a respective location of each of the one or more second regions of interest.

The method can further include generating first region of interest symbology indicating the one or more first regions of interest based on the location of imaging within the subject body in the second imaging modality and combining the first region of interest symbology with one or more invasive study images generated by the second imaging modality to generate one or more enhanced invasive study images.

In some implementations, generating the first region of interest symbology indicating the one or more first regions of interest based on the location of imaging within the subject body in the second imaging modality includes aligning a location of the one or more first regions of interest with the location of imaging within the subject body, and associating each element of the first region of interest symbology with a respective location of each of the one or more first regions of interest.

Some implementations can include a system comprising one or more processors coupled to a computer-readable medium having stored thereon software instructions that, when executed by the one or more processors, cause the one or more processors to perform operations. The operations can include obtaining one or more first images generated by a first imaging modality, detecting one or more first regions of interest based on a first detection modality, and detecting one or more second regions of interest based on a second detection modality. The operations can also include determining a location of imaging within a subject body in a second imaging modality and generating second region of interest symbology indicating the one or more second regions of interest based on the location of imaging within the subject body in the second imaging modality. The operations can further include combining the second region of interest symbology with one or more invasive study images generated by the second imaging modality to generate one or more enhanced invasive study images and causing the one or more enhanced invasive study images to be displayed during an invasive imaging study of the second imaging modality.

In some implementations, the first imaging modality includes positron emission tomography, and wherein the one or more first images include positron emission tomography images. In some implementations, the one or more first regions of interest each include an area containing a suspected hard plaque. Some implementations can include CT and/or MRI imaging modalities. During the time of an invasive vascular procedure, intravascular ultrasound ca be used which could help identify soft plaque at risk of rupture. In this scenario it is possible for the IP to identify (e.g., via the ML model) areas of concern and overlay symbology corresponding to those areas back onto the invasive angiography image.

In some implementations, the one or more second regions of interest each include an area containing a suspected soft plaque. In some implementations, the second detection modality includes a machine learning model trained to predict regions within an artery containing a suspected soft plaque with an increased risk of rupture. In some cases, there may be a soft plaque that is indolent in that it may calcify and become hard and benign, or it may become inflamed or bleed which could result in rupture, heart attack, stroke, or cold extremity.

In some implementations, generating second region of interest symbology indicating the one or more second regions of interest based on the location of imaging within the subject body in the second imaging modality includes aligning a location of the one or more second regions of interest with the location of imaging within the subject body, and associating each element of the second region of interest symbology with a respective location of each of the one or more second regions of interest.

The operations can also include generating first region of interest symbology indicating the one or more first regions of interest based on the location of imaging within the subject body in the second imaging modality and combining the first region of interest symbology with one or more invasive study images generated by the second imaging modality to generate one or more enhanced invasive study images.

In some implementations, generating first region of interest symbology indicating the one or more first regions of interest based on the location of imaging within the subject body in the second imaging modality includes aligning a location of the one or more first regions of interest with the location of imaging within the subject body, and associating each element of the first region of interest symbology with a respective location of each of the one or more first regions of interest.

Some implementations can include a computer-readable medium having stored thereon software instructions that, when executed by one or more processors, cause the one or more processors to perform operations. The operations can include obtaining one or more first images generated by a first imaging modality and detecting one or more first regions of interest based on a first detection modality. The operations can also include detecting one or more second regions of interest based on a second detection modality and determining a location of imaging within a subject body in a second imaging modality. The operations can further include generating second region of interest symbology indicating the one or more second regions of interest based on the location of imaging within the subject body in the second imaging modality and combining the second region of interest symbology with one or more invasive study images generated by the second imaging modality to generate one or more enhanced invasive study images. The operations can also include causing the one or more enhanced invasive study images to be displayed during an invasive imaging study of the second imaging modality.

In some implementations, the first imaging modality includes positron emission tomography, and wherein the one or more first images include positron emission tomography images. In some implementations, the one or more first regions of interest each include an area containing a suspected hard plaque.

In some implementations, the one or more second regions of interest each include an area containing a suspected soft plaque.

### BRIEF DESCRIPTION OF THE DRAWINGS

FIG. 1 is a block diagram of an example enhanced imaging system and associated environment which may be used for one or more implementations described herein.

FIG. 2 is a block diagram of an example enhanced imaging system including extended reality and an associated environment which may be used for one or more implementations described herein.

FIG. 3 is a flowchart showing an example enhanced invasive study imaging process in accordance with some implementations.

FIG. 4 is a diagram showing an example enhanced invasive study imaging graphical user interface in accordance with some implementations.

FIG. 5 is a block diagram of an example computing device which may be used for one or more implementations described herein.

### DETAILED DESCRIPTION

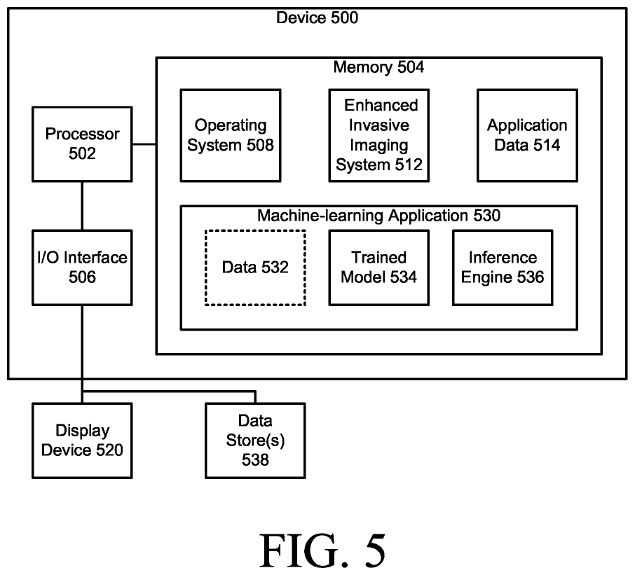

Some implementations include enhanced invasive study imaging methods and systems. When performing enhanced invasive study imaging functions, it may be helpful for a system to suggest regions of potential interest (or elevated risk) within images of a body that is the subject of the invasive study and/or to make predictions about risk to the study subject associated with regions of potential interest. To make predictions or suggestions, a probabilistic model (or other model as described below in conjunction with FIG. 5) can be used to make an inference (or prediction) about aspects of regions of potential interest such as potential risk to the study subject. Accordingly, it may be helpful to make an inference regarding a probability that a given region is of potential interest (for example, a given region includes a soft arterial plaque). Other aspects can be predicted or suggested as described below.

The inference based on the probabilistic model can include predicting regions of potential interest in accordance with image (or other data) analysis and confidence score as inferred from the probabilistic model. The probabilistic model can be trained with data including previous invasive study, non-invasive study, healthcare professional notations, and/or pathology data. Some implementations can include generating visual (or other) indications of regions of potential interest in a subject based on invasive and non-invasive study data of the subject.

The systems and methods provided herein may overcome one or more deficiencies of some conventional invasive study imaging systems and methods. For example, some conventional invasive study imaging systems may provide information about hard or calcified plaques but may not provide information on other areas of potential interest such as regions where a soft plaque may be present.

The example systems and methods described herein may overcome one or more of the deficiencies of conventional invasive study imaging systems to provide users with automated indications. A technical problem of some conventional invasive study imaging systems may be that such systems do not provide enhanced invasive study images in real-time (or near real-time) as described herein or may not suggest regions of potential interest based on non-invasive data and/or predict regions of potential interest based on non-invasive study data processed by a machine learning model to predict potential risk areas not associated only with hard plaques.

Particular implementations may realize one or more of the following advantages. An advantage of enhanced invasive study imaging and visualization based on methods and systems described herein is that the images and visualizations are based on processed non-invasive imaging study data and confidence, combined with invasive study images. Yet another advantage is that the methods and systems described herein can dynamically learn new thresholds (e.g., for confidence scores, etc.) and provide suggestions for regions of potential interest that match the new thresholds. The systems and methods presented herein automatically provide region of potential interest and/or risk predictions that are more likely to be accepted by users and that likely are more accurate.

Some implementations can be configured to provide relevant information on initial imaging such as PET, MRI, or dual energy CT, especially with intravenous contrast on board, for interpretation by machine learning or better evaluated with graphical or image manipulation (e.g., via a heat map or the like) where the most important areas to biopsy could be isolated and then depicted on the second imaging modality. Hybrid biopsy with MRI overlay onto US has been used for prostate cancer. However, some implementations use symbology to help better define the best place to biopsy, which could also be aided by a machine learning model. Specifically, for breast cancer, such an implementation could provide a significant improvement over conventional techniques as often what is biopsied is not as aggressive as what the cancer really is and finding the most aggressive cancer to sample could be aided by this implementation.

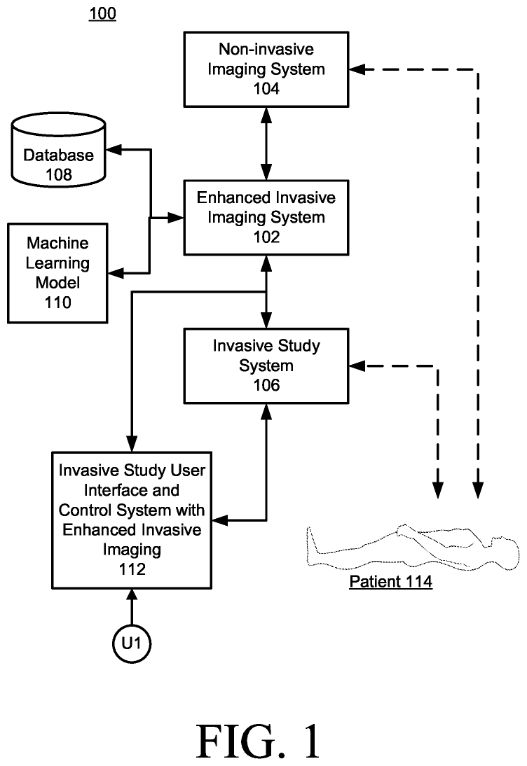

FIG. 1 is a block diagram of an example enhanced imaging system and associated environment 100 which may be used for one or more implementations described herein. The environment 100 can include an enhanced invasive imaging system 102, a non-invasive imaging system 104, an invasive study system 106, a database 108, a machine learning model 110, and an invasive study user interface and control system 112.

Some implementations can provide a way for radiologists, interventional radiologists, cardiologists, vascular surgeons and cardio-vascular surgeons to better identify potential soft plaques in blood vessels or arteries. Hard plaques, which are typically stable, can lead to narrowing or calcification of arteries but don't usually lead to strokes or heart attacks. On the other hand, soft plaques, which can be unstable, can become inflamed and rupture, which can lead to heart attack and strokes. Thus, providing a system that can help detect and visualize soft plaques during an invasive study of veins or arteries can permit a healthcare professional to place a stent or perform other suitable therapy to help stabilize the soft plaque to help prevent adverse events caused by the soft plaque.

In operation, non-invasive study data of a subject patient 114 body can be obtained via non-invasive imaging system 104 such as a positron emission tomography (PET) system, computed tomography (CT), MRI, PET-CT, PET-MRI, x-ray/fluoroscopy, or other non-invasive imaging systems. The non-invasive system 104 can generate data that can be used to help determine the location of plaques and nature (e.g., hard or soft) of those plaques. For example, in some implementations, soft plaques can be identified anatomically with ultrasound (US) (e.g., both external devices such as over the skin and intravascular devices—some small enough to enter coronary arteries). In some implementations, CT scans can be used to anatomically identify soft plaques. In some implementations, MRI can also be used to identify soft plaques. From a function standpoint, certain MRI sequences and PET scans using NaF radiotracer can be used to determine if there is active inflammation and instability which could result in rupture and clot formation leading to heart attack or stroke. In another example, some implementations can include dual energy CT scans to differentiate between different plaque types, such as stable soft and high risk soft.

In addition to one or more imaging technologies or techniques, a machine learning (ML) aspect of the disclosed subject matter can be configured (or trained) to identify soft plaques in general. Also, the ML model can be used to identify soft plaques with high-risk features of rupture. Further, the ML model can be trained to determine where anatomically the ROI is during a procedure. The ML model can be trained to both identify an ROI and display the location during the procedure. The ML model can be trained to identify a soft plaque, a hard plaque, and distinguish between the two. It will be appreciated that new imaging procedures, techniques, and methods are always being invented and could be utilized within the context of the disclosed subject matter where applicable.

In addition to identifying soft and hard plaques and their location, some implementations can include a capability of training the ML model to detect small aneurisms, which could be detected and the ROI localized at the time of surgery. Also, in some implementations, cancer could be detected, and a ML model trained to help overlay onto other images for image guided biopsy.

The non-invasive imaging system 104 data can be used as an input to the enhanced invasive imaging system 102. The non-invasive imaging system 104 can also receive data from the invasive study system 106, which can include a cardiac catheterization system or the like. The invasive study system 106 data can include image data, position data, and other data. Some implementations of the enhanced invasive imaging system 102 can take PET data, for example, and overlay the PET data with invasive study images to help guide a cardiologist during invasive study via catheterization. For example, some implementations can generate a 3-D rendering from image data to resemble what a cardiologist would see in real time to provide a hybrid “road map” to place a stent where there may not be a blockage (such as a hard plaque) but where the area may rupture due to a soft plaque.

To determine regions of potential interest or elevated risk (such as a predicted soft plaque region), the enhanced invasive imaging system 102 can include a database 108 and a machine learning model 110 trained to predict location of soft plaques or other elevated risk areas that might not be visible (or as visible as other features such as a hard plaque or calcification) during an invasive study.

The enhanced invasive imaging system 102 can generate enhanced imagery including indications of first regions (e.g., hard plaques) and second regions (e.g., soft plaques) based on a location of the imaging apparatus such as a catheter within the subject's body. The enhanced images can be provided to the invasive study user interface and control system 112 for display or presentation to a user. Controls on the invasive study user interface and control system 112 can be used to activate the enhanced imaging system and used to select types of symbology shown in the user interface (e.g., first regions, second regions, or both).

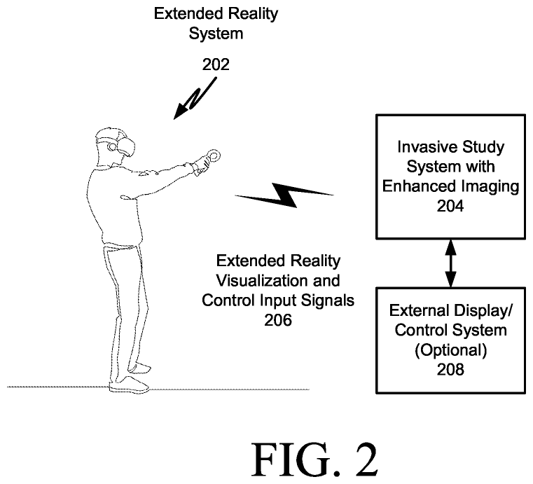

FIG. 2 is a block diagram of an example enhanced imaging system including extended reality and an associated environment which may be used for one or more implementations described herein. In addition to a traditional display system described above in connection with FIG. 1, some implementations can include an extended reality (XR) system 202 (e.g., augmented reality (AR), mixed reality (MR), virtual reality (VR), or the like) or, in general, any real-and-virtual combined environments and human-machine interactions generated by computer technology. The XR system 202 can communicate with an invasive study system with enhanced imaging system 204 (or other connected system), where the XR system 202 receives imagery or other data to present to a user within the XR environment. The user can provide control input to the invasive study system with enhanced imaging system 204 via movement of handheld controls, gestures, voice input, or the like. The presentation of enhanced invasive study data can include presentation of visual cues or imagery (including region of interest symbology), audio cues, and/or tactile or haptic feedback cues.

The invasive study system with enhanced imaging system 204, can be an integrated system including components similar to a combination of 102 and 106 shown in FIG. 1. In addition to the XR display 202, the system optionally can include an external display and controls 208 for use by a user not wearing or utilizing the extended reality system 202.

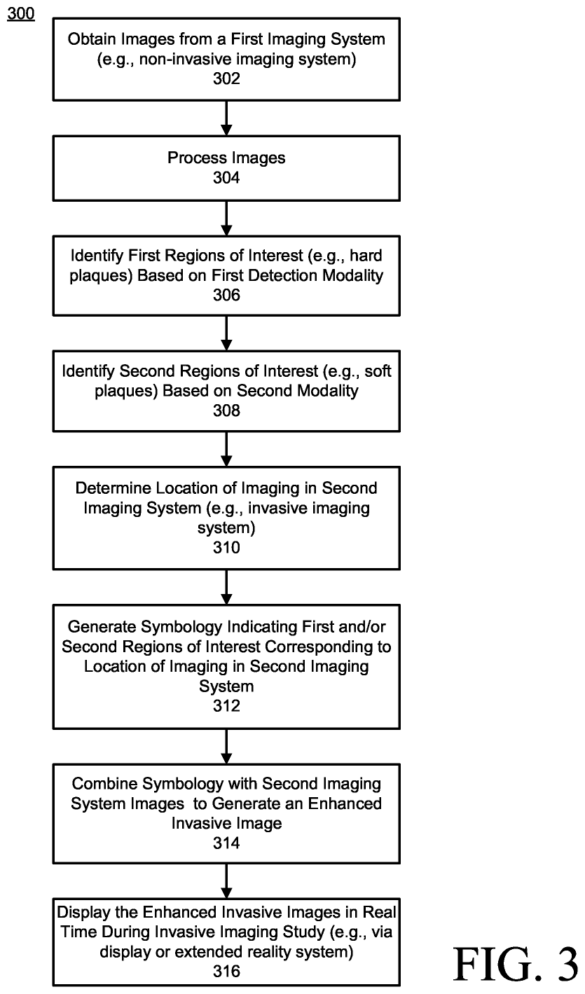

FIG. 3 is a flowchart showing an example enhanced invasive study imaging process 300 in accordance with some implementations. The process 300 begins at 302 where images are obtained from a first imaging system using a first imaging modality (e.g., a non-invasive imaging study system such as a PET or CT scanner or a hybrid PET-CT system, or MRI, PET-CT, PET-MRI, x-ray/fluoroscopy, or other now known or later developed suitable technique). Processing continues to 304.

At 304, the first images from the first imaging modality are optionally processed (e.g., subjected to image processing techniques to normalize the images, recognize objects within the images, align the images, etc.). Processing continues to 306.

At 306, first regions of interest are detected using a first detection modality. For example, a hard plaque detection operation is performed on the processed images. Processing continues to 308.

At 308, second regions of interest are detected using a second detection modality. For example, a model trained to identify soft plaques at risk for rupture, for example, can be used to perform a soft plaque detection operation on the processed images. Processing continues to 310.

At 310, a location of imaging is determined. For example, a location of a catheter of an invasive imaging study system within the subject's body being studied is obtained. Invasive procedures (such as arterial catheterization) are typically performed under fluoroscopy—essentially an x-ray video. A needle is used to access an artery, usually in the leg or arm, and through the needle a wire is placed. The needle is removed and then over the wire a small thin tube or catheter is placed. Then the wire is removed and a thicker one is put in place. A contrast agent that can be seen on x-ray is injected through the catheter and allows the operator to see the extent of the vascular tree. Now the operator can advance the catheter over the wire a little, then the wire, then the catheter, and so on. Essentially, the operator can inch their way to the area they need to operate on. Different catheters and wires exist which allow the operator to maneuver around corners, enter a side branch, or traverse a narrow vessel. Imaging technology exists, called a road map, which can save the image with contrast and overlay the image of the wire, so the operator doesn't need to keep injecting contrast. It should be noted that some people are allergic to contrast and air/CO2 can be used for such patients.

Some implementations can also include and be configured for different scenarios. For example, during a procedure for something else, such as treating a heart attack, imaging data from other studies can be used to identify an at-risk plaque and indicate to the operator that there are other areas that need treatment now. During the procedure, the user could decide to do real time imaging, such as with ultrasound (possible intravascular procedure or extravascular if at the carotid artery in the neck) and have the ML analyze those images to show where a vulnerable plaque is that may need treatment. Such a use would be similar to an add on procedure during the already scheduled operation. In some implementations, patients at high risk for vulnerable plaques, such as those with diabetes, hypertension, high cholesterol, prior stroke, or prior heart attack could undergo a screening type test to find vulnerable plaques which could then be used to guide the intervention if a problem is identified.

Based on the plaque morphology or other anatomic references, the ML model can be configured to coordinate the overlay between the current fluoroscopy image and a location of the vulnerable plaque ROI.

In some implementations, an MRI and an US can be used to guide intervention and wires/catheters. In some implementations, small intravascular robots can enter the body and be guided to sites of disease using imaging information and location information. Processing continues to 312.

At 312, symbology corresponding to one or more first region of interest and/or second region of interest is generated based on the location determined in 310. For example, if the invasive imaging catheter is at a given position within the subject's body, first and/or second symbology is generated based on whether there is a first and/or second region of interest at the location of the invasive imaging. Processing continues to 314.

At 314, the symbology is combined with the invasive study images to generate enhanced invasive study images showing first and/or second regions of interest when present. Processing continues to 316.

At 316, the enhanced invasive images are presented. For example, the enhanced invasive images can be displayed on a display or presented within an extended reality system or presented in any other suitable modality. In some implementations, in addition to the enhanced invasive study images, other information can be displayed to a user such as patient demographics and/or risk factors that may be relevant to a decision about using a therapy on a soft plaque or other elevated risk area. Further, in addition to the other information being presented to the operator, the patient demographic and/or risk factor information can be examined as part of the ML algorithm. For example, the ML model can include a false positive prediction of the soft plaque on imaging alone in a 20-year-old woman with no risk factors. This information could be used to alter the ML prediction from the image to say, low probability this is anything real (e.g., an artifact). On the other hand, a soft plaque seen in an 85-year-old hypertensive, diabetic, obese, and high-risk race/ethnicity (black or Hispanic) man would push the imaging ML data to say this is now very high risk and we need to do something. At the time of the procedure the overlay for the plaques can be configured to show most or all the areas of imaging concern and, in some implementations, present different colors corresponding to the risk level to suggest low, medium, or high risk based on other data put into the models.

In some implementations, the system can determine the thickness, length, surface contour, and location of one or more plaques. These features can factor into determining the vulnerability for rupture of a given soft plaque. These features can be in addition to other patient characteristics, such as smoking or inflammatory diseases. Some implementations can include an AR/MR/VR unit because the image could be created to show the topology/3D features to the operator in addition to utilizing the ML model prediction of risk. Thus, an operator can see in real time what the ML model is indicating and then make a clinical judgment of next steps. This information could be extracted as well. In some implementations, the model can generate generate a scoring system based on risk, which could be displayed or incorporated into the ROI color scheme of risk.

One or more of 302-316 can be repeated in whole or in part in the example order shown or in any other suitable order to accomplish an enhanced invasive imaging task.

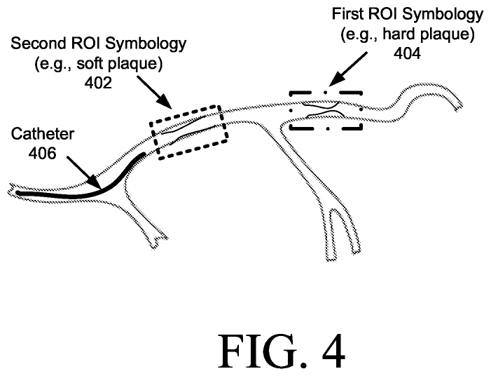

FIG. 4 is a diagram showing an example enhanced invasive study imaging graphical user interface in accordance with some implementations. In particular, FIG. 4 shows a second region of interest symbology 402 (e.g., indicating the location of a possible soft plaque), a first region of interest symbology 404 (e.g., indicating the location of a possible hard plaque), and location of a catheter 406.

The display of the first and second symbology can be performed in real-time or near real-time as an invasive study is conducted to help guide a practitioner in diagnosing and/or treating a patient. The first and second symbology can be the same or different (as shown in FIG. 4).

Further, while FIG. 4 shows a 2-D display, it will be appreciated that the visual information can be presented in one or more of a number of ways including an augmented reality (or extended reality) system. For example, a VR/AR/MR type platforms could allow for 3D views of the vessels or other target areas which could theoretically help with placement or better understanding of the anatomy.

In addition to a visual display, there are other types of indications or warnings that can be provided to the user such as audio, tactile, or haptic feedback. In a tactile implementation, a wireless device can vibrate on the hand, or the VR/AR/MR headset can vibrate. It will be hard to have the wire be tactile as it is likely in a vulnerable position in the body. We could also have the display change color when the catheter is in position. There are usually small dots that can be seen on Xray as part of the catheter delivery system for something like a stent. In some implementations, when images are registered with the ROI the different feedback mechanisms can turn on. Some implementations can include a cold, warm, warmer, hot type feature that can be configured to help guide users in difficult to reach places.

In some implementations, an enhanced invasive study imaging system can include one or more server systems. Each server system can communicate with a network, for example. Each server system can include a server device and a database (e.g., 102, 108, and 110) or other data store or data storage device. An enhanced invasive study imaging network environment also can include one or more client devices, e.g., 112 and 202, which may communicate with each other and/or with a server system via the network. The network can be any type of communication network, including one or more of the Internet, local area networks (LAN), wireless networks, switch or hub connections, etc. In some implementations, the network can include peer-to-peer communication between devices, e.g., using peer-to-peer wireless protocols.

For ease of illustration, FIG. 1 shows one block for enhanced invasive study imaging system 102, database 108, machine learning model 110, user interface and control system 112, non-invasive imaging system 104 and invasive imaging system 106. These blocks can be separate systems or one or more of the blocks can be integrated into a single system. Some blocks (e.g., 102, 104, and 106) may represent multiple systems, server devices, and network databases, and the blocks can be provided in different configurations than shown. For example, the enhanced invasive study imaging system 102 can represent multiple systems that can communicate with other systems via the network. In some examples, database 108 and/or other storage devices can be provided in server system block(s) that are separate from or integrated with other devices. Also, there may be any number of client devices. Each client device can be any type of electronic device, e.g., desktop computer, laptop computer, portable or mobile device, camera, cell phone, smart phone, tablet computer, television, TV set top box or entertainment device, wearable devices (e.g., display glasses or goggles, head-mounted display (HMD), wristwatch, headset, armband, jewelry, etc.), virtual reality (VR) and/or augmented reality (AR) enabled devices, personal digital assistant (PDA), media player, game device, etc. Some client devices may also have a local database similar to database 108 or other storage. In other implementations, an enhanced invasive study imaging environment may not have all of the components shown and/or may have other elements including other types of elements instead of, or in addition to, those described herein.

In various implementations, end-users (e.g., U1 and others) may communicate with enhanced invasive study imaging system 102 and/or each other using respective client devices. In some examples, users may interact with each other via applications running on respective client devices and/or enhanced invasive study imaging system 102, and/or via a network service, e.g., an image sharing service, a messaging service, a social network service or other type of network service, implemented on enhanced invasive study imaging system 102. For example, respective client devices 112 and others may communicate data to and from one or more server systems (e.g., enhanced invasive study imaging system 102). In some implementations, the enhanced invasive study imaging system 102 may provide appropriate data to the client devices such that each client device can receive communicated content or shared content uploaded to the enhanced invasive study imaging system 102 and/or network service. In some examples, the users can interact via audio or video conferencing, audio, video, or text chat, or other communication modes or applications. In some examples, the network service can include any system allowing users to perform a variety of communications, form links and associations, upload and post shared content such as images, image compositions (e.g., albums that include one or more images, image collages, videos, etc.), audio data, and other types of content, receive various forms of data, and/or perform enhanced invasive study imaging-related functions. For example, the network service can allow a user to send messages to particular or multiple other users, form social links in the form of associations to other users within the network service, group other users in user lists, friends lists, or other user groups, post or send content including text, images, image compositions, audio sequences or recordings, or other types of content for access by designated sets of users of the network service, participate in live video, audio, and/or text videoconferences or chat with other users of the service, etc. In some implementations, a “user” can include one or more programs or virtual entities, as well as persons that interface with the system or network.

A user interface can enable display of enhanced invasive study imaging images, image compositions, data, and other content as well as communications, privacy settings, notifications, and other data on client devices or server devices. Such an interface can be displayed using software on the client device, software on the server device, and/or a combination of client software and server software executing on server device, e.g., application software or client software in communication with enhanced invasive study imaging system 102. The user interface can be displayed by a display device of a client device or server device, e.g., a display screen, projector, etc. In some implementations, application programs running on a server system can communicate with a client device to receive user input at the client device and to output data such as visual data, audio data, etc. at the client device.

In some implementations, enhanced invasive study imaging system 102 and/or one or more client devices can provide enhanced invasive study imaging functions.

Various implementations of features described herein can use any type of system and/or service. Any type of electronic device can make use of features described herein. Some implementations can provide one or more features described herein on client or server devices disconnected from or intermittently connected to computer networks.

FIG. 5 is a block diagram of an example device 500 which may be used to implement one or more features described herein. In one example, device 500 may be used to implement a client device, e.g., devices 102-106 or 112 shown in FIG. 1. In some implementations, device 500 may be used to implement a client device, a server device, or a combination of the above. Device 500 can be any suitable computer system, server, or other electronic or hardware device as described above.

One or more methods described herein (e.g., 300) can be run in a standalone program that can be executed on any type of computing device, a program run on a web browser, a mobile application (“app”) run on a mobile computing device (e.g., cell phone, smart phone, tablet computer, wearable device (wristwatch, armband, jewelry, headwear, virtual reality goggles or glasses, augmented reality goggles or glasses, head mounted display, etc.), laptop computer, etc.).

In one example, a client/server architecture can be used, e.g., a mobile computing device (as a client device) sends user input data to a server device and receives from the server the final output data for output (e.g., for display). In another example, all computations can be performed within the mobile app (and/or other apps) on the mobile computing device. In another example, computations can be split between the mobile computing device and one or more server devices.

In some implementations, device 500 includes a processor 502, a memory 504, and I/O interface 506. Processor 502 can be one or more processors and/or processing circuits to execute program code and control basic operations of the device 500. A “processor” includes any suitable hardware system, mechanism or component that processes data, signals or other information. A processor may include a system with a general-purpose central processing unit (CPU) with one or more cores (e.g., in a single-core, dual-core, or multi-core configuration), multiple processing units (e.g., in a multiprocessor configuration), a graphics processing unit (GPU), a field-programmable gate array (FPGA), an application-specific integrated circuit (ASIC), a complex programmable logic device (CPLD), dedicated circuitry for achieving functionality, a special-purpose processor to implement neural network model-based processing, neural circuits, processors optimized for matrix computations (e.g., matrix multiplication), or other systems.

In some implementations, processor 502 may include one or more co-processors that implement neural-network processing. In some implementations, processor 502 may be a processor that processes data to produce probabilistic output, e.g., the output produced by processor 502 may be imprecise or may be accurate within a range from an expected output. Processing need not be limited to a particular geographic location or have temporal limitations. For example, a processor may perform its functions in “real-time,” “offline,” in a “batch mode,” etc. Portions of processing may be performed at different times and at different locations, by different (or the same) processing systems. A computer may be any processor in communication with a memory.

Memory 504 is typically provided in device 500 for access by the processor 502 and may be any suitable processor-readable storage medium, such as random-access memory (RAM), read-only memory (ROM), Electrically Erasable Read-only Memory (EEPROM), Flash memory, etc., suitable for storing instructions for execution by the processor, and located separate from processor 502 and/or integrated therewith. Memory 504 can store software operating on the server device 500 by the processor 502, including an operating system 408, machine-learning application 530, enhanced invasive imaging application 512, and application data 514. Other applications may include applications such as a data display engine, web hosting engine, image display engine, notification engine, social networking engine, etc. In some implementations, the machine-learning application 530 and enhanced invasive imaging application 512 can each include instructions that enable processor 502 to perform functions described herein, e.g., some or all of the methods of FIG. 3.

The machine-learning application 530 can include one or more NER implementations for which supervised and/or unsupervised learning can be used. The machine learning models can include multi-task learning based models, residual task bidirectional LSTM (long short-term memory) with conditional random fields, statistical NER, etc. The Device can also include an enhanced invasive imaging application 512 as described herein and other applications. One or more methods disclosed herein can operate in several environments and platforms, e.g., as a stand-alone computer program that can run on any type of computing device, as a web application having web pages, as a mobile application (“app”) run on a mobile computing device, etc.

In various implementations, machine-learning application 530 may utilize Bayesian classifiers, support vector machines, neural networks, or other learning techniques. In some implementations, machine-learning application 530 may include a trained model 534, an inference engine 536, and data 532. In some implementations, data 532 may include training data, e.g., data used to generate trained model 534. For example, training data may include any type of data suitable for training a model for enhanced invasive imaging application tasks, such as images, labels, thresholds, etc. associated with enhanced invasive imaging described herein. Training data may be obtained from any source, e.g., a data repository specifically marked for training, data for which permission is provided for use as training data for machine-learning, etc. In implementations where one or more users permit use of their respective user data to train a machine-learning model, e.g., trained model 534, training data may include such user data. In implementations where users permit use of their respective user data, data 532 may include permitted data.

In some implementations, data 532 may include collected data such as non-invasive and invasive imaging. In some implementations, training data may include synthetic data generated for the purpose of training, such as data that is not based on user input or activity in the context that is being trained, e.g., data generated from simulated non-invasive or invasive studies, computer-generated images, etc. In some implementations, machine-learning application 530 excludes data 532. For example, in these implementations, the trained model 534 may be generated, e.g., on a different device, and be provided as part of machine-learning application 530. In various implementations, the trained model 534 may be provided as a data file that includes a model structure or form, and associated weights. Inference engine 536 may read the data file for trained model 534 and implement a neural network with node connectivity, layers, and weights based on the model structure or form specified in trained model 534.

Machine-learning application 530 also includes a trained model 534. In some implementations, the trained model 534 may include one or more model forms or structures. For example, model forms or structures can include any type of neural-network, such as a linear network, a deep neural network that implements a plurality of layers (e.g., “hidden layers” between an input layer and an output layer, with each layer being a linear network), a convolutional neural network (e.g., a network that splits or partitions input data into multiple parts or tiles, processes each tile separately using one or more neural-network layers, and aggregates the results from the processing of each tile), a sequence-to-sequence neural network (e.g., a network that takes as input sequential data, such as words in a sentence, frames in a video, etc. and produces as output a result sequence), etc.

The model form or structure may specify connectivity between various nodes and organization of nodes into layers. For example, nodes of a first layer (e.g., input layer) may receive data as input data 532 or application data 514. Such data can include, for example, images, e.g., when the trained model is used for enhanced invasive imaging functions. Subsequent intermediate layers may receive as input output of nodes of a previous layer per the connectivity specified in the model form or structure. These layers may also be referred to as hidden layers. A final layer (e.g., output layer) produces an output of the machine-learning application. For example, the output may be a set of labels for an image, an indication that one or more areas of an image are of interest, etc. depending on the specific trained model. In some implementations, model form or structure also specifies a number and/or type of nodes in each layer.

In different implementations, the trained model 534 can include a plurality of nodes, arranged into layers per the model structure or form. In some implementations, the nodes may be computational nodes with no memory, e.g., configured to process one unit of input to produce one unit of output. Computation performed by a node may include, for example, multiplying each of a plurality of node inputs by a weight, obtaining a weighted sum, and adjusting the weighted sum with a bias or intercept value to produce the node output.

In some implementations, the computation performed by a node may also include applying a step/activation function to the adjusted weighted sum. In some implementations, the step/activation function may be a nonlinear function. In various implementations, such computation may include operations such as matrix multiplication. In some implementations, computations by the plurality of nodes may be performed in parallel, e.g., using multiple processors cores of a multicore processor, using individual processing units of a GPU, or special-purpose neural circuitry. In some implementations, nodes may include memory, e.g., may be able to store and use one or more earlier inputs in processing a subsequent input. For example, nodes with memory may include long short-term memory (LSTM) nodes. LSTM nodes may use the memory to maintain “state” that permits the node to act like a finite state machine (FSM). Models with such nodes may be useful in processing sequential data, e.g., words in a sentence or a paragraph, frames in a video, speech or other audio, etc.

In some implementations, trained model 534 may include embeddings or weights for individual nodes. For example, a model may be initiated as a plurality of nodes organized into layers as specified by the model form or structure. At initialization, a respective weight may be applied to a connection between each pair of nodes that are connected per the model form, e.g., nodes in successive layers of the neural network. For example, the respective weights may be randomly assigned, or initialized to default values. The model may then be trained, e.g., using data 532, to produce a result.

For example, training may include applying supervised learning techniques. In supervised learning, the training data can include a plurality of inputs (e.g., a set of images) and a corresponding expected output for each input (e.g., one or more labels for each image representing aspects of a project corresponding to the images such as services or products needed or recommended). Based on a comparison of the output of the model with the expected output, values of the weights are automatically adjusted, e.g., in a manner that increases a probability that the model produces the expected output when provided similar input.

In some implementations, training may include applying unsupervised learning techniques. In unsupervised learning, only input data may be provided and the model may be trained to differentiate data, e.g., to cluster input data into a plurality of groups, where each group includes input data that are similar in some manner. For example, the model may be trained to identify enhanced invasive imaging task labels that are associated with images and/or select thresholds for enhanced invasive imaging recommendations.

In another example, a model trained using unsupervised learning may cluster words based on the use of the words in data sources. In some implementations, unsupervised learning may be used to produce knowledge representations, e.g., that may be used by machine-learning application 530. In various implementations, a trained model includes a set of weights, or embeddings, corresponding to the model structure. In implementations where data 532 is omitted, machine-learning application 530 may include trained model 534 that is based on prior training, e.g., by a developer of the machine-learning application 530, by a third-party, etc. In some implementations, trained model 534 may include a set of weights that are fixed, e.g., downloaded from a server that provides the weights.

Machine-learning application 530 also includes an inference engine 536. Inference engine 536 is configured to apply the trained model 534 to data, such as application data 514, to provide an inference. In some implementations, inference engine 536 may include software code to be executed by processor 502. In some implementations, inference engine 536 may specify circuit configuration (e.g., for a programmable processor, for a field programmable gate array (FPGA), etc.) enabling processor 502 to apply the trained model. In some implementations, inference engine 536 may include software instructions, hardware instructions, or a combination. In some implementations, inference engine 536 may offer an application programming interface (API) that can be used by operating system 508 and/or enhanced invasive imaging application 512 to invoke inference engine 536, e.g., to apply trained model 534 to application data 514 to generate an inference.

Machine-learning application 530 may provide several technical advantages. For example, when trained model 534 is generated based on unsupervised learning, trained model 534 can be applied by inference engine 536 to produce knowledge representations (e.g., numeric representations) from input data, e.g., application data 514. For example, a model trained for enhanced invasive imaging tasks may produce predictions and confidences for given input information about an invasive imaging study. A model trained for suggesting enhanced invasive imaging indications may produce a suggestion for one or more areas of potential interest, or a model for automatic estimating or evaluation of an enhanced invasive imaging study may automatically estimate likelihood of areas of potential interest (e.g., soft plaques within veins or arteries) based on input images or other information. In some implementations, such representations may be helpful to reduce processing cost (e.g., computational cost, memory usage, etc.) to generate an output (e.g., a suggestion, a prediction, a classification, etc.). In some implementations, such representations may be provided as input to a different machine-learning application that produces output from the output of inference engine 536.

In some implementations, knowledge representations generated by machine-learning application 530 may be provided to a different device that conducts further processing, e.g., over a network. In such implementations, providing the knowledge representations rather than the images may provide a technical benefit, e.g., enable faster data transmission with reduced cost. In another example, a model trained for enhanced invasive imaging may produce an enhanced invasive imaging signal for one or more images being processed by the model.

In some implementations, machine-learning application 530 may be implemented in an offline manner. In these implementations, trained model 534 may be generated in a first stage and provided as part of machine-learning application 530. In some implementations, machine-learning application 530 may be implemented in an online manner. For example, in such implementations, an application that invokes machine-learning application 530 (e.g., operating system 508, one or more of enhanced invasive imaging application 512 or other applications) may utilize an inference produced by machine-learning application 530, e.g., provide the inference to a user, and may generate system logs (e.g., if permitted by the user, an action taken by the user based on the inference; or if utilized as input for further processing, a result of the further processing). System logs may be produced periodically, e.g., hourly, monthly, quarterly, etc. and may be used, with user permission, to update trained model 534, e.g., to update embeddings for trained model 534.

In some implementations, machine-learning application 530 may be implemented in a manner that can adapt to particular configuration of device 500 on which the machine-learning application 530 is executed. For example, machine-learning application 430 may determine a computational graph that utilizes available computational resources, e.g., processor 502. For example, if machine-learning application 530 is implemented as a distributed application on multiple devices, machine-learning application 530 may determine computations to be carried out on individual devices in a manner that optimizes computation. In another example, machine-learning application 530 may determine that processor 502 includes a GPU with a particular number of GPU cores (e.g., 1000) and implement the inference engine accordingly (e.g., as 1000 individual processes or threads).

In some implementations, machine-learning application 530 may implement an ensemble of trained models. For example, trained model 534 may include a plurality of trained models that are each applicable to same input data. In these implementations, machine-learning application 530 may choose a particular trained model, e.g., based on available computational resources, success rate with prior inferences, etc. In some implementations, machine-learning application 530 may execute inference engine 536 such that a plurality of trained models is applied. In these implementations, machine-learning application 530 may combine outputs from applying individual models, e.g., using a voting-technique that scores individual outputs from applying each trained model, or by choosing one or more particular outputs. Further, in these implementations, machine-learning application may apply a time threshold for applying individual trained models (e.g., 0.5 ms) and utilize only those individual outputs that are available within the time threshold. Outputs that are not received within the time threshold may not be utilized, e.g., discarded. For example, such approaches may be suitable when there is a time limit specified while invoking the machine-learning application, e.g., by operating system 508 or one or more other applications, e.g., enhanced invasive imaging application 512.

In different implementations, machine-learning application 530 can produce different types of outputs. For example, machine-learning application 530 can provide representations or clusters (e.g., numeric representations of input data), labels (e.g., for input data that includes images, documents, etc.), phrases or sentences (e.g., descriptive of an image or video, suitable for use as a response to an input sentence, suitable for use to determine context during a conversation, etc.), images (e.g., generated by the machine-learning application in response to input), audio or video (e.g., in response an input video, machine-learning application 530 may produce an output video with a particular effect applied, e.g., rendered in a comic-book or particular artist's style, when trained model 534 is trained using training data from the comic book or particular artist, etc. In some implementations, machine-learning application 530 may produce an output based on a format specified by an invoking application, e.g., operating system 508 or one or more applications, e.g., enhanced invasive imaging application 512. In some implementations, an invoking application may be another machine-learning application. For example, such configurations may be used in generative adversarial networks, where an invoking machine-learning application is trained using output from machine-learning application 530 and vice-versa.

Any of software in memory 504 can alternatively be stored on any other suitable storage location or computer-readable medium. In addition, memory 504 (and/or other connected storage device(s)) can store one or more messages, one or more taxonomies, electronic encyclopedia, dictionaries, thesauruses, knowledge bases, message data, grammars, user preferences, and/or other instructions and data used in the features described herein. Memory 504 and any other type of storage (magnetic disk, optical disk, magnetic tape, or other tangible media) can be considered “storage” or “storage devices.”

I/O interface 506 can provide functions to enable interfacing the server device 500 with other systems and devices. Interfaced devices can be included as part of the device 500 or can be separate and communicate with the device 500. For example, network communication devices, storage devices (e.g., memory and/or database 106), and input/output devices can communicate via I/O interface 506. In some implementations, the I/O interface can connect to interface devices such as input devices (keyboard, pointing device, touchscreen, microphone, camera, scanner, sensors, etc.) and/or output devices (display devices, speaker devices, printers, motors, etc.).

Some examples of interfaced devices that can connect to I/O interface 506 can include one or more display devices 520 and one or more data stores 538 (as discussed above). The display devices 520 that can be used to display content, e.g., a user interface of an output application as described herein. Display device 520 can be connected to device 400 via local connections (e.g., display bus) and/or via networked connections and can be any suitable display device. Display device 520 can include any suitable display device such as an LCD, LED, or plasma display screen, CRT, television, monitor, touchscreen, 3-D display screen, or other visual display device. For example, display device 520 can be a flat display screen provided on a mobile device, multiple display screens provided in a goggles or headset device, or a monitor screen for a computer device.

The I/O interface 506 can interface to other input and output devices. Some examples include one or more cameras which can capture images. Some implementations can provide a microphone for capturing sound (e.g., as a part of captured images, voice commands, etc.), audio speaker devices for outputting sound, or other input and output devices.

For ease of illustration, FIG. 5 shows one block for each of processor 502, memory 504, I/O interface 506, and software blocks 508, 512, and 530. These blocks may represent one or more processors or processing circuitries, operating systems, memories, I/O interfaces, applications, and/or software modules. In other implementations, device 500 may not have all of the components shown and/or may have other elements including other types of elements instead of, or in addition to, those shown herein. While some components are described as performing blocks and operations as described in some implementations herein, any suitable component or combination of components of environment 100, device 500, similar systems, or any suitable processor or processors associated with such a system, may perform the blocks and operations described.

In some implementations, logistic regression can be used for personalization (e.g., personalizing enhanced invasive imaging suggestions based on a user's pattern of enhanced invasive imaging activity). In some implementations, the prediction model can be handcrafted including hand selected labels and thresholds. The mapping (or calibration) from ICA space to a predicted precision within the enhanced invasive imaging space can be performed using a piecewise linear model.

In some implementations, the enhanced invasive imaging system could include a machine-learning model (as described herein) for tuning the system (e.g., selecting one or more areas of interest or concern and corresponding thresholds) to potentially provide improved accuracy. Inputs to the machine learning model can include ICA labels, an image descriptor vector that describes appearance and includes semantic information about enhanced invasive imaging. Example machine-learning model input can include labels for a simple implementation and can be augmented with descriptor vector features for a more advanced implementation. Output of the machine-learning module can include a prediction of one or more areas of interest or concern within invasive study images.

One or more methods described herein (e.g., method 300) can be implemented by computer program instructions or code, which can be executed on a computer. For example, the code can be implemented by one or more digital processors (e.g., microprocessors or other processing circuitry), and can be stored on a computer program product including a non-transitory computer readable medium (e.g., storage medium), e.g., a magnetic, optical, electromagnetic, or semiconductor storage medium, including semiconductor or solid state memory, magnetic tape, a removable computer diskette, a random access memory (RAM), a read-only memory (ROM), flash memory, a rigid magnetic disk, an optical disk, a solid-state memory drive, etc. The program instructions can also be contained in, and provided as, an electronic signal, for example in the form of software as a service (SaaS) delivered from a server (e.g., a distributed system and/or a cloud computing system). Alternatively, one or more methods can be implemented in hardware (logic gates, etc.), or in a combination of hardware and software. Example hardware can be programmable processors (e.g., Field-Programmable Gate Array (FPGA), Complex Programmable Logic Device), general purpose processors, graphics processors, Application Specific Integrated Circuits (ASICs), and the like. One or more methods can be performed as part of or component of an application running on the system, or as an application or software running in conjunction with other applications and operating system.

One or more methods described herein can be run in a standalone program that can be run on any type of computing device, a program run on a web browser, a mobile application (“app”) run on a mobile computing device (e.g., cell phone, smart phone, tablet computer, wearable device (wristwatch, armband, jewelry, headwear, goggles, glasses, etc.), laptop computer, etc.). In one example, a client/server architecture can be used, e.g., a mobile computing device (as a client device) sends user input data to a server device and receives from the server the final output data for output (e.g., for display). In another example, all computations can be performed within the mobile app (and/or other apps) on the mobile computing device. In another example, computations can be split between the mobile computing device and one or more server devices.

Although the description has been described with respect to particular implementations thereof, these particular implementations are merely illustrative, and not restrictive. Concepts illustrated in the examples may be applied to other examples and implementations.

Note that the functional blocks, operations, features, methods, devices, and systems described in the present disclosure may be integrated or divided into different combinations of systems, devices, and functional blocks. Any suitable programming language and programming techniques may be used to implement the routines of particular implementations. Different programming techniques may be employed, e.g., procedural or object-oriented. The routines may execute on a single processing device or multiple processors. Although the steps, operations, or computations may be presented in a specific order, the order may be changed in different particular implementations. In some implementations, multiple steps or operations shown as sequential in this specification may be performed at the same time.

What is claimed is:

1. A computer-implemented method comprising:
obtaining one or more first images generated by a first imaging modality;
detecting one or more first regions of interest based on a first detection modality;
detecting one or more second regions of interest based on a second detection modality;
determining a location of imaging within a subject body in a second imaging modality;
generating second region of interest symbology indicating the one or more second regions of interest based on the location of imaging within the subject body in the second imaging modality;
combining the second region of interest symbology with one or more invasive study images generated by the second imaging modality to generate one or more enhanced invasive study images; and
causing the one or more enhanced invasive study images to be displayed during an invasive imaging study of the second imaging modality.

2. The computer-implemented method of claim 1, wherein the first imaging modality includes one of positron emission tomography, CT, MRI, US, PET-CT, PET-MRI, and x-ray/fluoroscopy, and wherein the one or more first images include images generated by positron emission tomography, CT, MRI, US, PET-CT, PET-MRI, or x-ray/fluoroscopy, respectively.

3. The computer-implemented method of claim 1, wherein the one or more first regions of interest each include an area containing a suspected hard vascular plaque.

4. The computer-implemented method of claim 1, wherein the one or more second regions of interest each include an area containing a suspected soft vascular plaque at risk for rupture.

5. The computer-implemented method of claim 1, wherein the second detection modality includes a machine learning model trained to predict regions within an artery containing a suspected soft plaque.

6. The computer-implemented method of claim 1, wherein generating the second region of interest symbology indicating the one or more second regions of interest based on the location of imaging within the subject body in the second imaging modality includes:
aligning a location of the one or more second regions of interest with the location of imaging within the subject body; and
associating each element of the second region of interest symbology with a respective location of each of the one or more second regions of interest.

7. The computer-implemented method of claim 1, further comprising:
generating first region of interest symbology indicating the one or more first regions of interest based on the location of imaging within the subject body in the second imaging modality; and
combining the first region of interest symbology with one or more invasive study images generated by the second imaging modality to generate one or more enhanced invasive study images.

8. The computer-implemented method of claim 7, wherein generating the first region of interest symbology indicating the one or more first regions of interest based on the location of imaging within the subject body in the second imaging modality includes:
aligning a location of the one or more first regions of interest with the location of imaging within the subject body; and
associating each element of the first region of interest symbology with a respective location of each of the one or more first regions of interest.

9. A system comprising:
one or more processors coupled to a computer-readable medium having stored thereon software instructions that, when executed by the one or more processors, cause the one or more processors to perform operations including:
obtaining one or more first images generated by a first imaging modality;
detecting one or more first regions of interest based on a first detection modality;
detecting one or more second regions of interest based on a second detection modality;
determining a location of imaging within a subject body in a second imaging modality;
generating second region of interest symbology indicating the one or more second regions of interest based on the location of imaging within the subject body in the second imaging modality;
combining the second region of interest symbology with one or more invasive study images generated by the second imaging modality to generate one or more enhanced invasive study images; and
causing the one or more enhanced invasive study images to be displayed during an invasive imaging study of the second imaging modality.

10. The system of claim 9 wherein the first imaging modality includes one of positron emission tomography, CT, MRI, US, PET-CT, and PET-MRI, and wherein the one or more first images include images generated by positron emission tomography, CT, MRI, US, PET-CT, or PET-MRI, respectively.

11. The system of claim 9, wherein the one or more first regions of interest each include an area containing a suspected hard vascular plaque.

12. The system of claim 9, wherein the one or more second regions of interest each include an area of interest for biopsy or surgery.

13. The system of claim 9, wherein the second detection modality includes a machine learning model trained to predict regions within an artery containing a suspected soft plaque.

14. The system of claim 9, wherein generating second region of interest symbology indicating the one or more second regions of interest based on the location of imaging within the subject body in the second imaging modality includes:
aligning a location of the one or more second regions of interest with the location of imaging within the subject body; and
associating each element of the second region of interest symbology with a respective location of each of the one or more second regions of interest.

15. The system of claim 9, further comprising:
generating first region of interest symbology indicating the one or more first regions of interest based on the location of imaging within the subject body in the second imaging modality; and
combining the first region of interest symbology with one or more invasive study images generated by the second imaging modality to generate one or more enhanced invasive study images.

16. The system of claim 15, wherein generating first region of interest symbology indicating the one or more first regions of interest based on the location of imaging within the subject body in the second imaging modality includes:
aligning a location of the one or more first regions of interest with the location of imaging within the subject body; and
associating each element of the first region of interest symbology with a respective location of each of the one or more first regions of interest.

17. A computer-readable medium having stored thereon software instructions that, when executed by one or more processors, cause the one or more processors to perform operations including:
obtaining one or more first images generated by a first imaging modality;
detecting one or more first regions of interest based on a first detection modality;
detecting one or more second regions of interest based on a second detection modality;
determining a location of imaging within a subject body in a second imaging modality;
generating second region of interest symbology indicating the one or more second regions of interest based on the location of imaging within the subject body in the second imaging modality;
combining the second region of interest symbology with one or more invasive study images generated by the second imaging modality to generate one or more enhanced invasive study images; and
causing the one or more enhanced invasive study images to be displayed during an invasive imaging study of the second imaging modality.

18. The computer-readable medium of claim 17, wherein the first imaging modality includes one of positron emission tomography, CT, MRI, US, PET-CT, and PET-MRI, and wherein the one or more first images include images generated by positron emission tomography, CT, MRI, US, PET-CT, or PET-MRI, respectively.

19. The computer-readable medium of claim 17, wherein the one or more first regions of interest each include an area containing a suspected hard plaque.

20. The computer-readable medium of claim 17, wherein the one or more second regions of interest each include an area of interest for biopsy or surgery.

21. The computer-implemented method of claim 1, wherein the one or more second regions of interest each include an area of interest for biopsy or surgery.
